(19) United States
(12) Patent Application Publication
(10) Pub. No.: US 2018/0265874 A1
(43) Pub. Date: Sep. 20, 2018

(54) METHODS FOR TREATING AND/OR PREVENTING A TUMOR GROWTH, INVASION AND/OR METASTASIS

(71) Applicants: GLOBAL BIOPHARMA, INC., TAIPEI CITY (TW); TAIPEI MEDICAL UNIVERSITY, TAIPEI (TW)

(72) Inventors: HUIE LEE, TAIPEI (TW); YU-JU HUANG, TAIPEI CITY (TW)

(21) Appl. No.: 15/518,088

(22) PCT Filed: Oct. 9, 2015
(86) PCT No.: PCT/US15/54960

§ 371 Date: Apr. 10, 2017

Related U.S. Application Data
(60) Provisional application No. 62/062,202, filed on Oct. 10, 2014.

(51) Int. Cl. C12N 15/113; A61K 45/06; A61K 31/713; C07K 16/28; A61K 39/395

(52) U.S. Cl. CPC C12N 15/1138; A61K 45/06; A61K 31/713; C07K 16/2827; A61K 39/39558; C12N 2310/14; C07K 2317/76; C12N 2310/111; C12N 2310/531; C07K 2317/31; C07K 2317/24; C07K 2317/21; C12N 2310/122

(57) ABSTRACT

The invention relates to a method for treating and/or preventing a tumor growth, invasion and/or metastasis by inhibiting an overexpression of PD-L1 in the subject via an autocrine loop. The invention found that PD-L1-modulated p21 and VEGF-C expression via the TGFβ1/SMAD4 pathway is responsible for the cancer growth, invasion and metastasis.

### FIELD OF THE INVENTION

The invention relates to a method for treating and/or preventing a tumor growth and/or metastasis in a subject. Particularly, the invention relates to a method for treating and/or preventing a tumor growth and/or metastasis by inhibiting autocrine loop mechanism of action and knockdowning an expression of PD-L1 in the subject.

### BACKGROUND OF THE INVENTION

Programmed death-1 (PD-1) is a costimulatory molecule that provides an inhibitory signal in T cell activation. In contrast, PD-L1 is abundant in human carcinoma of lung, ovary and colon and in melanomas. PD-L1 secreted from cancer cells were bound to its receptor PD-1 on T cell membrane to promote tumor progression and metastasis via blocking tumor immune surveillance. A large body of literatures indicated that the number of CD+4 and CD+8 tumor infiltrating lymphocytes (TILs) in tumor parts may be reduced by PD-L1 overexpression to promote tumor malignancy and poor prognosis in human carcinomas including lung cancer. Two ligands for PD-1, PD-L1 (B7-H1) and PD-L2 (B7-DC) have been identified and those are cell-surface glycoprotein belonging to the B7 family. The primary result of PD-1 ligation by its ligands is to inhibit signaling downstream of the T cell Receptor (TCR). Therefore, signal transduction via PD-1 usually provides a suppressive or inhibitory signal to the T cell that results in decreased T cell proliferation or other reduction in T cell activation. PD-1 signaling is thought to require binding to a PD-1 ligand in close proximity to a peptide antigen presented by major histocompatibility complex (MHC), which is bound to the TCR. PD-L1, also known as B7 homolog 1 (B7-H1) or CD274, is the predominant PD-1 ligand causing inhibitory signal transduction in T cells. PD-L1 is expressed at low levels on immune cells such as B cells, dendritic cells, macrophages and T cells and is up regulated following activation. PD-L1 is also expressed on non-lymphoid organs such as endothelial cells, heart, lung, pancreas, muscle, keratinocytes and placenta. The expression within non lymphoid tissues suggests that PD-L1 may regulate the function of self-reactive T and B cells as well as myeloid cells in peripheral tissues or may regulate inflammatory responses in the target organs. PD-L1 expression is mainly regulated by type 1 and 2 interferons which are major regulators of PD-L1 on endothelial and epithelial cells.

Konishi et al. first reported that fewer tumor infiltrating lymphocytes were observed in PD-L1-positive lung tumors than in PD-L1-negative lung tumors, suggesting that PD-L1 expressed from lung tumors may contribute to negative regulation in antitumor immune response in non-small cell lung cancer (NSCLC) (Konishi J, Yamazaki K, Azuma M, et al. B7-1 expression on non-small cell lung cancer cells and its relationship with tumor infiltrating lymphocytes and their PD-1 expression. Clin Cancer Res 2004; 10: 5094-100). PD-L1 is frequently found to be highly expressed in many human cancer types being upregulated in tumors by activation of key oncogenic pathways, such as PI3K, MAPK, and most strongly by upregulated by IFN-γ in the tumor microenvironment to cause an active antitumor T-cell response (Chen, T H, Huang, C C, Yeh K T, Chang S H, Sung W W, Cheng Y W and Lee H* (2012) Human papillomavirus 16 E6 oncoprotein associated with p53 inactivation in colorectal cancer. World J Gastroenterol, 18, 4051-4058). In HPV positive-head and neck squamous cell carcinoma (HNSCC) that are highly infiltrated with lymphocytes, PD-L1 expression on both tumors and CD68+ tumor-associated macrophages is located to sites of lymphocytes fronts, whereas the majority of CD8+ TIL inducers of PD-L1 expression were found in HPV-positive PD-L1(+) tumors. These results support the role of the PD-1:PD-L1 interaction in creating an immune-privileged site for initial viral infection and subsequent adaptive immune resistance once tumors are established and suggest a rationale for therapeutic blockade of this in patients with HPV-positive HNSCC (Lyford-Pike et al. 2013). The expression of PD-L1 in tumors has been reported to relate to tumor-infiltrating lymphocytes (TILs) and correlated with poor clinical prognosis in melanoma; in cancers of ovarian, breast, esophageal, kidney, gastric, pancreas, bladder, hepatocellular and squamous cell carcinoma of the head and neck and even in adult T-cell leukemia/lymphoma. In lung cancer, PD-L1 has been demonstrated that contribute to negative regulation in antitumor immune response; however, the postoperative survival was not associated with PD-L1 expression.

US20110177088 relates to a method of treatment of hematologic malignancies comprising the step of administering to a subject in need thereof a therapeutically effective amount of a ligand of PD1, wherein said ligand of PD1 is selected from the group consisting of PD-L1 or a fragment thereof which binds to PD1, PD-L2 or a fragment thereof which binds to PD1, and an anti-PD1 antibody or a fragment thereof which binds to PD1, and wherein the hematologic malignancy is selected from the group consisting of a chronic lymphocytic leukemia (CLL) of B-cell origin, a small lymphocytic lymphoma (SLL) of B-cell origin, a multiple myeloma, an acute B cell leukemia and a mantle cell lymphoma. US 20130149305 provides a soluble CD80 protein that interacts with programmed death ligand 1 (PD-L1) and thereby inhibiting the interaction of PD-L1 with T-cell expressed programmed death 1 (PD1) receptor, and thus, minimizing PD-L1 mediated immune suppression. The tumor-infiltrating T cells have been found to upregulate immunosuppressive pathways, such as PD-L1, in a paracrine fashion on tumor cells. Particularly, Vamsidhar Velcheti et al. indicates that PD-L1 expression was significantly associated with tumor-infiltrating lymphocytes and a study on patients with non-small cell lung cancer showed that greater PD-L1 protein and mRNA expression is associated with increased local lymphocytic infiltrate and longer survival (Vamsidhar Velcheti et al., Programmed Death Ligand-1 Expression in Non-small Cell Lung Cancer, Laboratory Investigation (2014) 94, 107-116).

However, the mechanisms regulating PD-L1 expression in tumor cells are not known and the exact sequence of events within the tumor microenvironment that culminates in tumor regression is also unknown. There is a need to explore the relationship between PD-L1 expression and tumor malignancy.

### SUMMARY OF THE INVENTION

The invention provides method for treating and/or preventing a tumor growth, invasion and/or metastasis in a subject, comprising administering a PD-1 expression inhibitor to a subject to knockdown an expression of PD-L1 in the subject via an autocrine loop. In one embodiment, the administration of PD-L1 enhances TGF-β1 singnaling pathway, preferably, TGF β1/SMAD4 signaling pathway. In one embodiment, the enhancement of TGF β1/SMAD4 signaling pathway increases p21 expression to inhibit tumor proliferation and/or decreases VEGF-C expression to inhibit tumor metastasis. In one embodiment, the tumor is a virus-associated tumor. Preferably, the virus is HPV, HIV, EBV, HBV, CMV or HCV. For example, the tumor is HPV-, HIV-, HCV-, EBV- or HBV-associated cancer, cancers of reproductive organs, renal cancer, colon cancer, breast cancer, kidney cancer, pancreatic cancer, colon cancer, large bowel cancer, lung cancer, liver cancer, brain tumor, gastric cancer, uterine cervix cancer, ovary cancer, prostate cancer, urinary bladder cancer, esophageal cancer, leukemia, lymphoma, fibrosarcoma, mastocytoma, or melanoma. In one embodiment, the PD-L1 expression inhibitor is a small interfering RNA (siRNA) of PD-L1, a small hairpin (sh)RNA of PD-L1 or an antisense RNA of PD-L1 or a monoclonal antibody against PD-L1. In one embodiment, the subject is a relapsed or refractory subject. Preferably, the subject is a mammal.

The invention provides a pharmaceutical combination, comprising a PD-L1 expression inhibitor in combination with a TGF-β1 expression inhibitor or VEGF expression inhibitor. In one embodiment, the PD-L1 expression inhibitor is a small interfering RNA (siRNA) of PD-L1, a small hairpin (sh)RNA of PD-L1, an antisense RNA of PD-L1, an anti-PD-L1 antibody or an antigen-binding fragment thereof. Preferably, the anti-PD-L1 antibody is chimeric, humanized, composite, human antibody or bispecific antibody. In another embodiment, the pharmaceutical combination further comprises a second anticancer agent or therapy.

### BRIEF DESCRIPTION OF THE DRAWING

FIGS. 1 A-H shows the effect of PD-L1 expression on the colony formation and doubling time in lung cancer cells. Endogenous HPV16-positive TL-1 and TL-2 cells which were established from pleural effusions of lung adenocarcinoma patients were transfected with a small hairpin RNA PD-L1 (shPD-L1) to silencing PD-L1 expression for 48 h. A549 and TL-4 cells were transfected with various doses of PD-L1 expression vector to overexpress PD-L1 for 48 h. After shPD-L1 or PD-L1 expression vector transfection, the colony formation efficacy and doubling time of these cells were evaluated by colony formation and MTT assay.

FIGS. 2 A-F show the effects of PD-L1 on soft-agar growth, invasion capability, and xenograft lung tumor formation. The soft-agar growth and invasion capability changed by PD-L1-knockdown in TL-1 and TL-2 cells or -overexpression in A549 and TL-4 cells were evaluated by soft-agar growth and invasion assay. Each two PD-L1-knockdown TL-1 and PD-L1 overexpressing TL-4 stable clones were used to inject into nude mice via tail vain. After 60 days, the numbers of lung tumor nodules in nude mice were counted and the tumors were further confirmed by H & E stain. The soft-agar growth colony and invasive cells on matrigel membrane in different cells with shPD-L1 or PD-L1 expression vector transfections were shown in upper panel). The lung tumor nodules and H & E stain were shown in middle panel. The changes of soft-agar growth, invasion capability and lung tumor nodules by PD-L1-knockdown or -overexpression were shown as the column.

FIGS. 3 A-H shows that decrease of TGF-β1 expression is responsible for PD-L1-mediated cell growth and invasion capability in mutant EGFR lung adenocarcinoma cells. PD-L1 expression in H1650 and H1975 cells was knocked down by its shRNA (5 μg) and then TGF-β1 were further silenced by two doses of shRNA. The expression of PD-L1, TGF-β1, VEGF-C, and p21 were evaluated by western blot. The change of cell growth and invasion capability by PD-L1 and/or TGF-β1 silencing was evaluated by MTT and Boyden chamber assay, respectively.

### DETAILED DESCRIPTION OF THE INVENTION

The invention is, in part, based on that the expression/overexpression of PD-L1 promotes cell proliferation and oncogenic potential in cancer cells. PD-L1 expressed from tumor cells could directly promote tumor malignancy and in turn result in poor outcome in cancer. Accordingly, PD-L1 expressed from tumors may promote tumor growth and metastasis via the autocrine loop and endogenous expression of PD-L1 in cancer cells promotes cell proliferation, growth, invasion and metastasis. Particularly, the invention found that PD-L1 expression is significantly higher in virus-infected cancer patients than in -non-infected cancer patients. The invention shows that patients with high PD-L1 tumors exhibited poorer outcomes compared with patients with low PD-L1 tumors. The studies in the invention demonstrates that PD-L1 promoted cell proliferation, colony formation, soft-agar growth, and xenograft tumor formation in lung cancer cells via autocrine loop. Furthermore, PD-L1-modulated p21 and VEGF-C expression via the TGFβ1/SMAD4 pathway is responsible for the tumor growth, invasion and metastasis. The elevated PD-L1 expression in cancer tumors is significantly associated with tumor aggressiveness such as invasion and metastasis, especially in HPV infected lung cancer patients.

Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. For example, the term “a cell” includes a plurality of cells, including mixtures thereof.

As used herein, the terms “tumor” and “cancer” are used interchangeably and refer to a malignant new growth of tissue that possesses no physiological function and arises from uncontrolled usually rapid cellular proliferation.

The terms “overexpress”, “overexpressed” and “overexpressing” as used herein interchangeably refer to a cancer or malignant cell that has measurably higher levels of PD-L1 on the surface compared to a normal cell of the same tissue type. Such overexpression may be caused by gene amplification or by increased transcription or translation. PD-L1 expression and overexpression can be measured using well know assays using for example ELISA, immunofluorescence, flow cytometry or radioimmunoassay on live or lysed cells. Alternatively, or additionally, levels of PD-L1 encoding nucleic acid molecules may be measured in the cell for example using fluorescent in situ hybridization, Southern blotting, or PCR techniques. PD-L1 is overexpressed when the level of PD-L1 on the surface of the cell is at least 1.2-fold higher when compared to the normal cell.

As used herein, the term “inhibition of TGF-1 signaling” means that TGF-1 fails to bind to the receptor, then Smad2 and Smad3 fail to undergo phosphorylation, thus failing to form a complex with Smad4, and as a result, the complex fails to translocate to the nucleus and regulate transcription.

As used herein, the tumor suppressor gene smad4 is synonymous with other designations for the same tumor suppressor gene that are known to those of skill in the art, including but not limited to madh4 and dpc4. As is conventional, the products of the expression of this gene is designated herein as SMAD4, which is synonymous with the corresponding other designations for the expression product of this gene that are known to those of skill in the art, including but not limited to MADH4 and DPC4.

As used herein, a “cancer cell” or a “tumor cell” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo.

As used herein, the term “pharmaceutically acceptable carrier”, refers to encompasses any of the standard pharmaceutical carriers, e.g., a phosphate buffered saline solution, water, and emulsions, such as an oil/water or water/oil emulsion, and various types of wetting agents. The compositions may also include stabilizers and preservatives and any of the above noted carriers with the additional proviso that they be acceptable for use in vivo. For examples of carriers, stabilizers and adjuvants, see Martin REMINGTON'S PHARM. SCI., 18th Ed., Mack Publ. Co., Easton, Pa. (1995), and in the “PHYSICIAN'S DESK REFERENCE”, 58th ed., Medical Economics, Montvale, N.J. (2004).

As used herein, the term “subject” is defined herein to include animals such as mammals, including, but not limited to, primates (e.g., humans), cows, sheep, goats, horses, dogs, cats, rabbits, rats, mice and the like. In specific embodiments, the subject is a human. The terms “subject” and “patient” are used interchangeably herein in reference, for example, to a mammalian subject, such as a human.

As used herein, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or an antibody or dosage form provided herein, with or without one or more additional active agent(s), after the diagnosis or onset of symptoms of the particular disease.

As used herein, the term “antibody” is meant to include both intact molecules as well as fragments thereof that include the antigen-binding site. These include, but not limited to, Fab and F(ab′)₂ fragments which lack the Fc fragment of an intact antibody, and a bi-specific antibody.

As used herein, the terms “prevent,” “preventing” and “prevention” refer to the prevention of the onset, recurrence or spread of a disease or disorder, or of one or more symptoms thereof. In certain embodiments, the terms refer to the treatment with or administration of a compound or an antibody or dosage form provided herein, with or without one or more other additional active agent(s), prior to the onset of symptoms, particularly to patients at risk of disease or disorders provided herein. The terms encompass the inhibition or reduction of a symptom of the particular disease. In this regard, the term “prevention” may be interchangeably used with the term “prophylactic treatment.

As used herein, the term “relapsed” refers to a situation where a subject, that has had a remission of cancer after a therapy, has a return of cancer cells.

As used herein, the term “refractory” or “resistant” refers to a circumstance where a subject, even after intensive treatment, has residual cancer cells in the body.

As used herein, the term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to.

As used herein, the term “anticancer agent” or “cancer therapeutic agent” is meant to include anti-proliferative agents and chemotherapeutic agents.

As used herein, the terms “co-administration” and “in combination with” include the administration of two or more therapeutic agents simultaneously, concurrently or sequentially within no specific time limits unless otherwise indicated. In one embodiment, the therapeutic agents are in the same composition or unit dosage form. In other embodiments, the therapeutic agents are in separate compositions or unit dosage forms.

In one aspect, the invention provides a method for treating and/or preventing a tumor growth, invasion and/or metastasis in a subject, comprising administering a PD-L1 expression inhibitor to a subject to knockdown an expression of PD-L1 in the subject via an autocrine loop. In other hand, the invention provide a use of a PD-L1 expression inhibitor in the manufacture of a medicament for treating and/or preventing a tumor growth, invasion and/or metastasis in a subject by knockdown an expression of PD-L1 in the subject via an autocrine loop.

The levels of TGFβ1, SMAD4, and p21 were markedly increased, but VEGF-C expression was decreased in PD-L1-knockdown tumor cells. The increase and decrease of these molecules were rescued by TGFβ1 silencing in PD-L1-knockdown tumor cells. More interestingly, PD-L1-modulated p21 and VEGF-C expression via the TGFβ1/SMAD4 pathway is responsible for the invasion capability in tumor cells. Accordingly, the invention provides a method for treating and/or preventing a tumor growth, invasion and/or metastasis in a subject, comprising administering a PD-L1 expression inhibitor and a TGF-β1 or VEGF expression inhibitor to a subject.

Transforming growth factor-β (TGF-β) plays a dual role in cell cycle arrest, apoptosis, homeostasis, wound healing and immune regulation. In the case of cancers, TGF-β signaling plays a context-dependent dual role, both as a tumor suppressor in early stage disease and as a tumor promoter in established cancers. Smad4, a tumor suppressor gene, is a central mediator in the signaling pathways of the TGF-beta superfamily. The SMAD pathway is the canonical signaling pathway of TGF-β family members. TGF-beta binds either to a type III receptor, which then presents TGF-β to a type II receptor, or TGF-β binds directly to type II receptors. Once activated by TGF-β, type II receptors recruit, bind, and transphosphorylate type I receptors which leads to the recruitment and phosphorylation of the intracellular effector proteins Smad2 and Smad3. Phosphorylated Smad2 and Smad3 subsequently bind to Smad4 and translocate to the nucleus to initiate gene expression. There is a strong correlation between malignant progression and loss of sensitivity to the antiproliferative effects of TGF-β which is frequently associated with reduced expression or inactivation of TGF-β receptors.

In one embodiment, the tumor is a virus-associated tumor. In one embodiment, the virus is HPV, HIV, EBV, HBV, CMV or HCV.

In one embodiment, the PD-L1 expression inhibitor used in the method of the invention is a small interfering RNA (siRNA) of PD-L1, a small hairpin (sh)RNA of PD-L1 or an antisense RNA of PD-L1 or a monoclonal antibody against PD-L1. Preferably, the target sequence of siRNA of PD-L1, shRNA of PD-L1 or antisense RNA of PD-L1 is GCTGCACTAATTGTCTATTGG (SEQ ID NO: 5).

In one embodiment, the subject is a relapsed or refractory subject. In one embodiment, the subject is a mammal. Preferably, the subject is a primate (e.g., human), a cow, a sheep, a goat, a horse, a dog, a cat, a rabbit, a rat or a mouse.

In one embodiment, the tumor or cancer includes, without limitation, HPV-, HIV-, HCV-, EBV-, CMV- or HBV-associated cancer, anal cancer, reproductive organ cancers (such as uterine cancer, ovarian cancer, endometrial cancer, cervical cancer, vaginal cancer, vulvar cancer and breast cancer), renal cancer, colon cancer, breast cancer, kidney cancer, pancreatic cancer, large bowel cancer, colon cancer, lung cancer, liver cancer, brain tumor, gastric cancer, uterine cervix cancer, ovary cancer, prostate cancer, urinary bladder cancer, esophageal cancer, leukemia, lymphoma, fibrosarcoma, mastocytoma, or melanoma. Preferably, the cancer is a leukemia, anal cancer, vulvar cancer, vaginal cancer, penile cancer, cervical cancer, head and neck cancer such as oropharyngeal cancer and cancer of the oral cavity, lung cancer, colon cancer, non-melanoma skin cancer, HPV-associated cancer, or liver cancer. Preferably, the cancer is a non-small-cell lung carcinoma (NSCLC), HPV-associated cancer.

In a further embodiment, the method of the invention disclosed herein comprises further administering a second anticancer agent.

In another aspect, the invention provides a pharmaceutical combination, comprising a PD-L1 expression inhibitor in combination with a TGF-β1 or VEGF expression inhibitor.

In one embodiment, the invention provides a pharmaceutical combination, comprising a PD-L1 expression inhibitor and a second anticancer agent. In another embodiment, the invention provides a pharmaceutical combination, comprising a PD-L1 expression inhibitor and TGF-β1 expression inhibitor or VEGF expression inhibitor optionally in combination with a second anticancer agent.

In one embodiment, the PD-L1 expression inhibitor is a small interfering RNA (siRNA) of PD-L1, a small hairpin (sh)RNA of PD-L1 or an antisense RNA of PD-L1, an anti-PD-L1 antibody, or an antigen-binding fragment thereof, that binds to a PD-1 protein. Preferably, the anti-PD-L1 antibody is chimeric, humanized, composite, human antibody or bispecific antibody.

Combination therapies may include a second anticancer agent. The PD-L1 antibody of the invention may also be administered together with a second anti-cancer agent, anti-TGFβ cytokine anti-VEGF monoclonal antibody, or a combination thereof.

The second anticancer agent as disclosed herein includes, but not limited to, an antimetabolite (e.g., 5-fluoro uracil, methotrexate, fludarabine, cytarabine (also known as cytosine arabinoside or Ara-C), and high dose cytarabine), antimicrotubule agent (e.g., vinca alkaloids, such as vincristine and vinblastine; and taxane, such as paclitaxel and docetaxel), alkylating agent (e.g., mechlorethamine, chlorambucil, cyclophosphamide, melphalan, melphalan, ifosfamide, carmustine, azacitidine, decitabine, busulfan, cyclophosphamide, dacarbazine, ifosfamide, and nitrosoureas, such as carmustine, lomustine, bischloroethylnitrosurea, and hydroxyurea), platinum agent (e.g., cisplatin, carboplatin, oxaliplatin, satraplatin (JM-216), and CI-973), anthracycline (e.g., doxorubicin and daunorubicin), antitumor antibiotic (e.g., mitomycin, bleomycin, idarubicin, adriamycin, daunomycin (also known as daunorubicin, rubidomycin, or cerubidine), and mitoxantrone), topoisomerase inhibitor (e.g., etoposide and camptothecin), purine antagonist or pyrimidine antagonist (e.g., 6-mercaptopurine, 5-fluorouracil, cytarabine, clofarabine, and gemcitabine), cell maturing agent (e.g., arsenic trioxide and tretinoin), DNA repair enzyme inhibitor (e.g., podophyllotoxine, etoposide, irinotecan, topotecan, and teniposide), enzyme that prevents cell survival (e.g., asparaginase and pegaspargase), histone deacetylase inhibitors (e.g., vorinostat), any other cytotoxic agents (e.g., estramustine phosphate, dexamethasone, prednimustine, and procarbazine), hormone (e.g., dexamethasone, prednisone, methylprednisolone, tamoxifen, leuprolide, flutamide, and megestrol), monoclonal antibody (e.g., gemtuzumab ozogamicin, alemtuzumab, rituximab, and yttrium-90-ibritumomab tiuxetan), immuno-modulator (e.g., thalidomide and lenalidomide), Bcr-Abl kinase inhibitor (e.g., AP23464, AZD0530, CGP76030, PD180970, SKI-606, imatinib, BMS354825 (dasatinib), AMN107 (nilotinib), and VX-680), hormone agonist or antagonist, partial agonist or partial antagonist, kinase inhibitor, surgery, radiotherapy (e.g., gamma-radiation, neutron bean radiotherapy, electron beam radiotherapy, proton therapy, brachytherapy, and systemic radioactive isotopes), endocrine therapy, biological response modifiers (e.g., interferons, interleukins, and tumor necrosis factor), hyperthermia and cryotherapy, and agents to attenuate any adverse effect (e.g., antiemetics). In one embodiment, the anticancer agent or cancer therapeutic agent is a cytotoxic agent, an anti-metabolite, an antifolate, an HDAC inhibitor such as MGCD0103 (a.k.a. N-(2-aminophenyl)-4-((4-(pyridin-3-yl)pyrimidin-2-ylamino)methyl)benzamid-e), a DNA intercalating agent, a DNA cross-linking agent, a DNA alkylating agent, a DNA cleaving agent, a topoisomerase inhibitor, a CDK inhibitor, a JAK inhibitor, an anti-angiogenic agent, a Bcr-Abl inhibitor, an HER2 inhibitor, an EGFR inhibitor, a VEGFR inhibitor, a PDGFR inhibitor, an HGFR inhibitor, an IGFR inhibitor, a c-Kit inhibitor, a Ras pathway inhibitor, a PI3K inhibitor, a multi-targeted kinase inhibitor, an mTOR inhibitor, an anti-estrogen, an anti-androgen, an aromatase inhibitor, a somatostatin analog, an ER modulator, an anti-tubulin agent, a vinca alkaloid, a taxane, an HSP inhibitor, a Smoothened antagonist, a telomerase inhibitor, a COX-2 inhibitor, an anti-metastatic agent, an immunosuppressant, a biologics such as antibodies and hormonal therapies.

The pharmaceutical combinations of the present invention may further comprise one or more pharmaceutically acceptable carriers, excipients, and other agents that are incorporated into formulations to provide improved transfer, delivery, tolerance, and the like (herein collectively referred to as “pharmaceutically acceptable carriers or diluents”). A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, 1998, J Pharm Sci Technol 52:238-311.

Accordingly, combinations/compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like.

The combinations/compositions of the present invention can be administered parenterally such as, for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampoules, disposable syringes or multiple dose vials made of glass or plastic.

Rectal administration includes administering the pharmaceutical combinations/compositions into the rectum or large intestine. This can be accomplished using suppositories or enemas. Suppository formulations can easily be made by methods known in the art. For example, suppository formulations can be prepared by heating glycerin to about 120° C., dissolving the pectin composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

The invention illustrates that PD-L1 expression is significantly higher in human papillomavirus (HPV) 16/18-infected patients than in HPV-non-infected lung cancer patients. Moreover, patients with high PD-L1 tumors exhibited poorer outcomes compared with patients with low PD-L1 tumors. Studies in the cell and animal models demonstrated that PD-L1 promoted cell proliferation, colony formation, soft-agar growth, and xenograft tumor formation in lung cancer via autocrine loop. The data herein from lung cancer patients shows that PD-L1 expression was higher in HPV-positive tumors than in HPV-negative tumors. Interestingly, patients with high PD-L1 expressing tumors exhibited shorter overall survival and relapse free survival periods than those with low PD-L1 expressing tumors. Surprisingly, the data herein from cell and animal model indicated that PD-L1 expressed from lung cancer cells can directly promote the soft-agar growth, invasiveness, and metastatic xenograft tumor formation.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example

### Materials and Methods

Study Population.

Lung tumor specimens were collected from 223 patients with primary lung cancer. All of these patients were admitted to the Department of Thoracic Surgery at Taichung Veteran's General Hospital, Taiwan, between 1998 and 2008. All patients were asked to submit a written informed consent based on a biology study approved by the institutional review board. None of the subjects had received radiation therapy or chemotherapy before surgery. Collected lung tumors have previously been analyzed for the presence of HPV16 and/or HPV18 E6 protein (16), and tumor types and stages were histologically determined according to the WHO (1981) classification. Pathology samples were processed for conventional histologic procedures.

Quantitative Real-Time RT-PCR.

Total RNA was prepared from lung cells and tumor specimens using TRIZOL reagent (Invitrogen). Total RNA (5 μg) was used in cDNA synthesis with random primers using Superscript III reverse transcriptase (Applied Biosystems). The resulting cDNA (1:20 dilution) was used to detect the expression of endogenous PD-L1 mRNA by qPCR. qPCR assays were performed at least in triplicate using the ABsolute qPCR SYBR Green ROX mix (Applied Biosystems, Foster City, Calif.) in a 7500HT real-time PCR system apparatus (Applied Biosystems, Foster City, Calif.). The primers used were as follows: (a) PD-L1, forward primer 5′-ACCTGACCTGCCGTCTAGAA-3′ (SEQ ID NO: 1) and reverse primer 5′-TCCACCACCCTGTTGCTGTA-3′ (SEQ ID NO: 2); (b) 18S rRNA, forward primer 5′-GTGAGCGATGGAACTTCGACTT-3′ (SEQ ID NO: 3) and reverse primer 5′-GGCGTTTGGAGTGGTAGAAATC-3′ (SEQ ID NO: 4). No-reverse-transcription (no-RT) controls were performed with 100 ng of total RNA from each individual sample as a template to ensure that amplification was not due to contamination with DNA. No signal could be detected in the no-RT control. Relative mRNA expression was calculated with the comparative Cₜ method (ΔΔCₜ). 18S rRNA was used for normalization.

Plasmids and Transfection.

The target sequence of the PD-L1-RNAi was GCTGCACTAATTGTCTATTGG (SEQ ID NO: 5). The RNAi template was cloned into the vector pCDNA-HU6 as described in Ann Surg Oncol, Epub Jun. 12, 2012. Plasmids containing the PD-L1 expression construct were constructed by cloning the full-length human PD-L1 cDNA (GenBank accession number NM 014143) into the pcDNA3.1 eukaryotic expression vector, which also expresses a neomycin (Neo) resistance gene. All transfection experiments were performed with TransFast transfection reagents (Promega) in accordance with the manufacturer's protocols.

Soft Agar Colony Formation Assay.

Cells (3000 per well) were cultured on a 6 well plate containing 1% base agar and 0.35% top agar in the medium described above and incubated at 37° C. for 21 days. Plates were stained with 0.005% crystal violet for 1 h. Colonies was counted using a dissecting microscope. Colony diameters larger than 100 μm were counted as 1 positive colony.

Boyden Chamber Assay.

A Boyden chamber with a pore size of 8 μm (Falcon) was used for in invasion assay. For the invasion assay, the upper side of the filter was covered with Matrigel (Becton Dickinson Labware). After 24 hours, cells on the upper side of the filter were removed and cells that adhered to the underside of the membrane were fixed in 95% ethanol and stained with 10% Giemsa dye. The number of migrated cells was counted using a fluorescence microscope (Olympus, Lake Success, N.Y.). Ten contiguous fields of each sample were examined to obtain a representative number of cells that migrated/invaded across the membrane. Each condition was assayed in triplicate.

Statistical Analysis.

Statistical analysis was performed using the SPSS statistical software program (Version 11.0 SPSS Inc., Chicago, Ill., USA). The association between PD-L1 and clinicopathologic variables was analyzed using the Pearson Chi-Square or Fisher's exact test as appropriate. The correlation between of PD-L1 and HPV was analyzed using Spearman's rank correlation. The survival curves were estimated by the Kaplan-Meier method and compared by the log-rank test. The Cox-regression model was used to perform univariate and multivariate analyses, including all the clinicopathologic features as covariates. We use the term of tumor status as T factor, nodal status as N factor, and metastatic status as M factor in tumor-node-metastasis classification, respectively. P value of <0.05 was considered as statistically significant.

### Example 1 Example 1 PD-L1 Promotes the Cell Proliferation, Colony Formation, Soft-Agar Growth and Invasion Capability in NSCLC Cells

The methods used to verify the effect of PD-L1 on tumor growth and metastasis in the cell model as follows: PD-L1 expression was silenced by a small hairpin (sh)RNA in high PD-L1 expressing cells; conversely, PD-L1 expression was ectopically expressed by its expression vector in low PD-L1 cells. The change of cell proliferation and invasion capability after the cells transfected with shRNA or expression vector of PD-L1 were evaluated by colony formation and Boyden chamber assay as compared with their control cells.

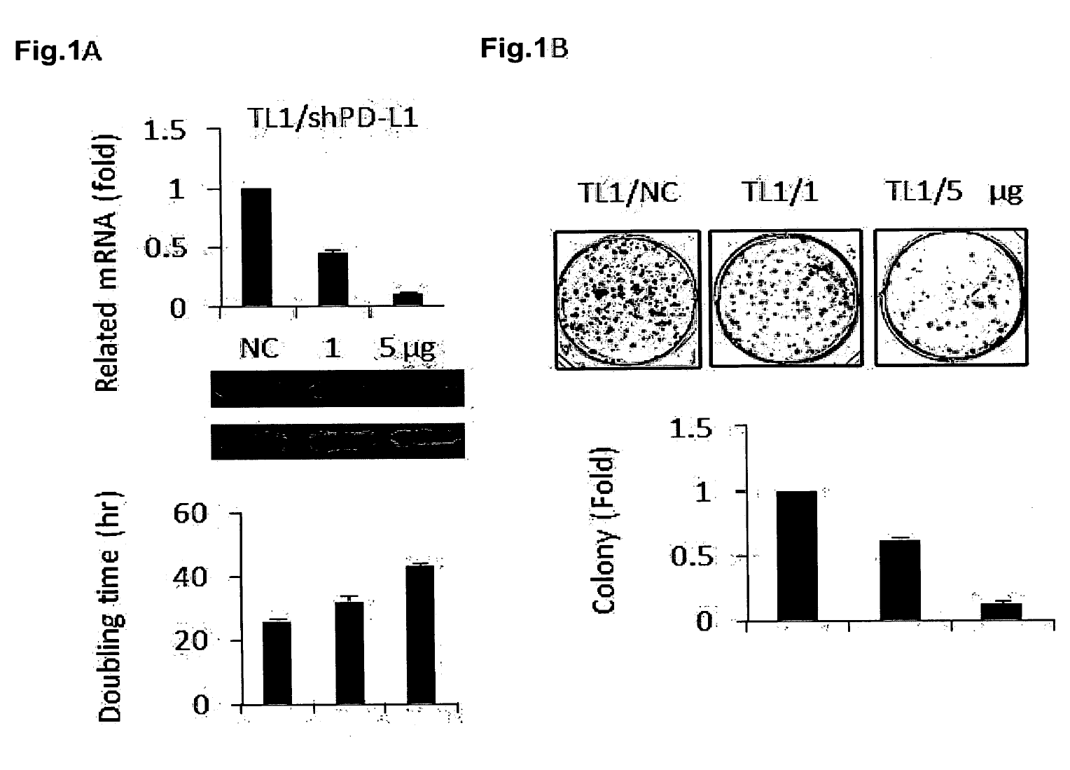

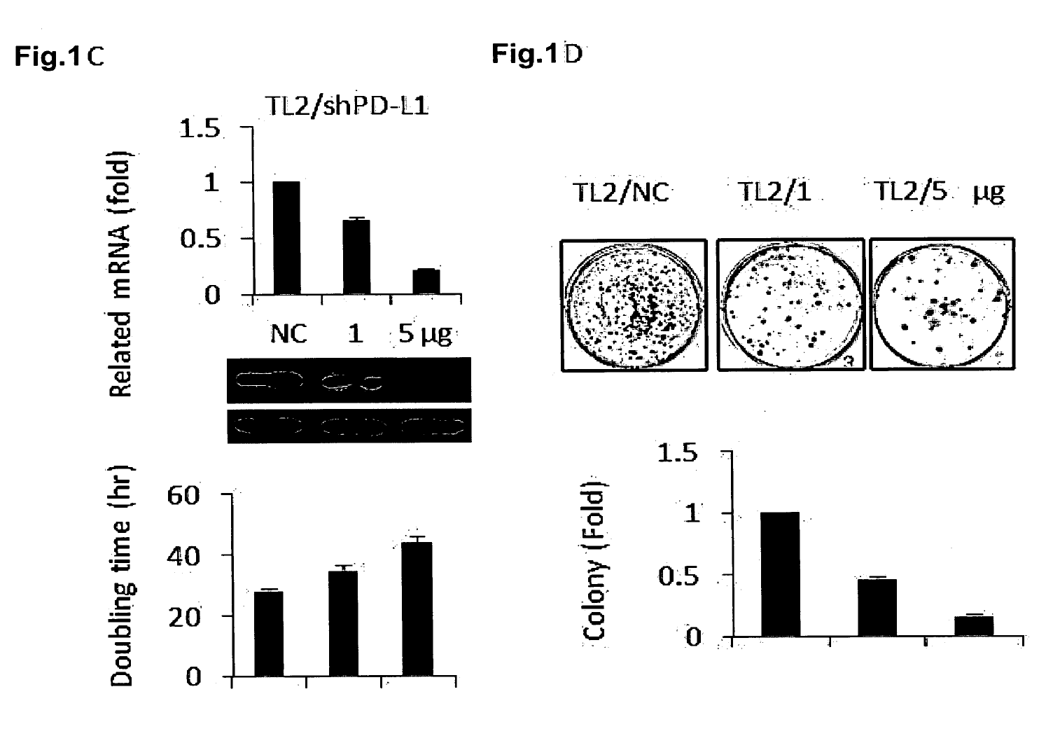

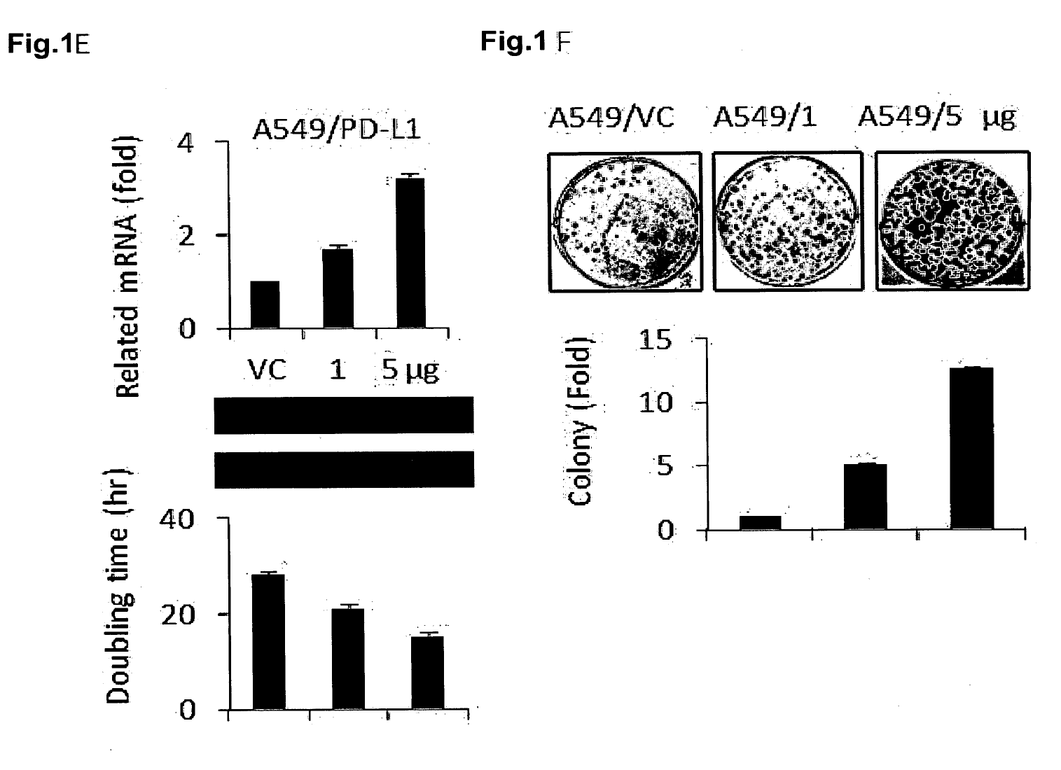

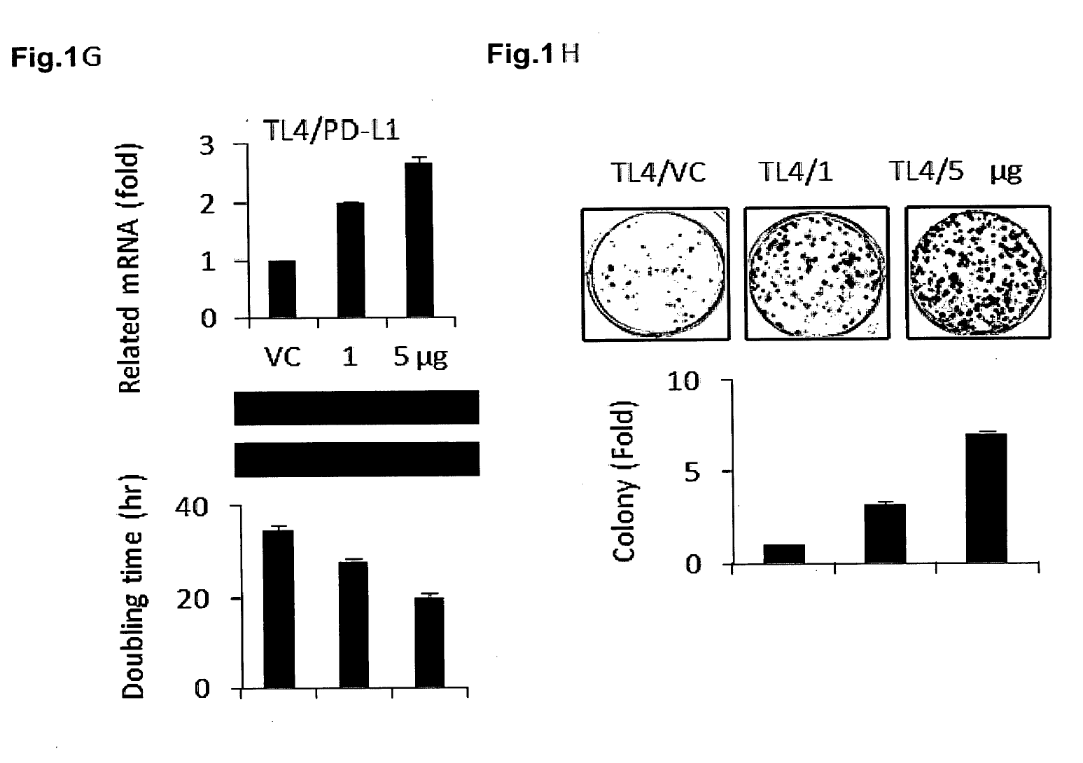

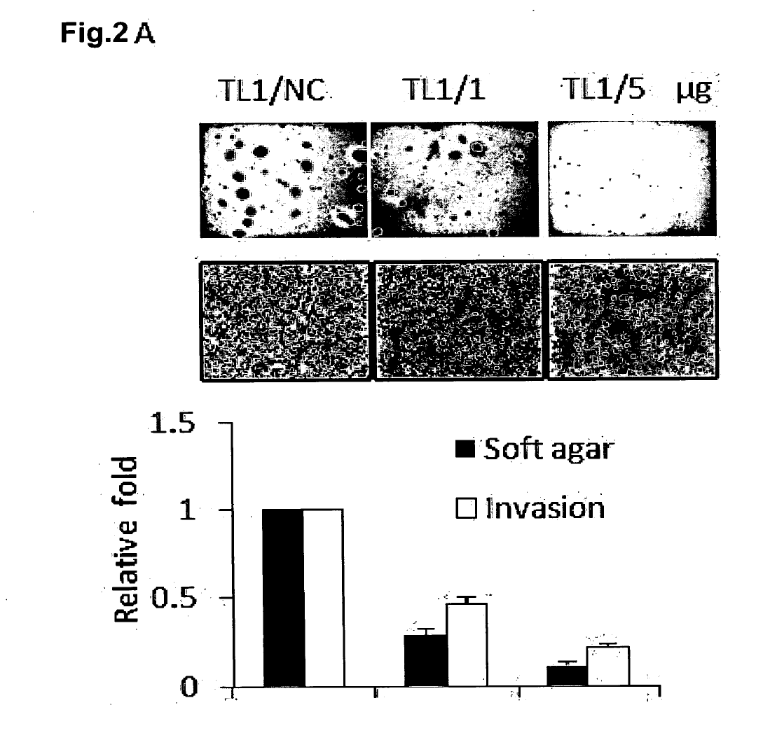

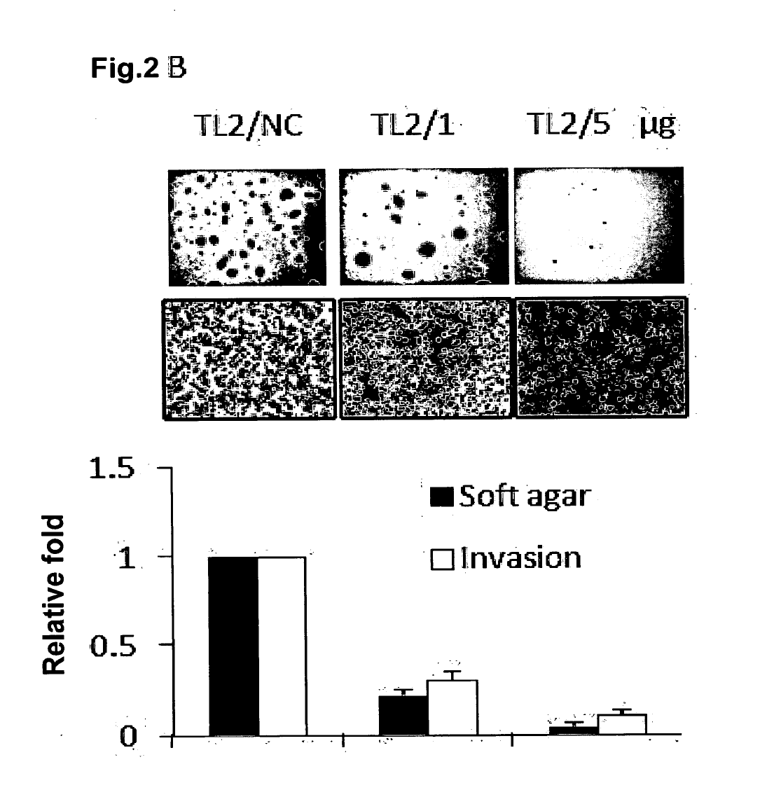

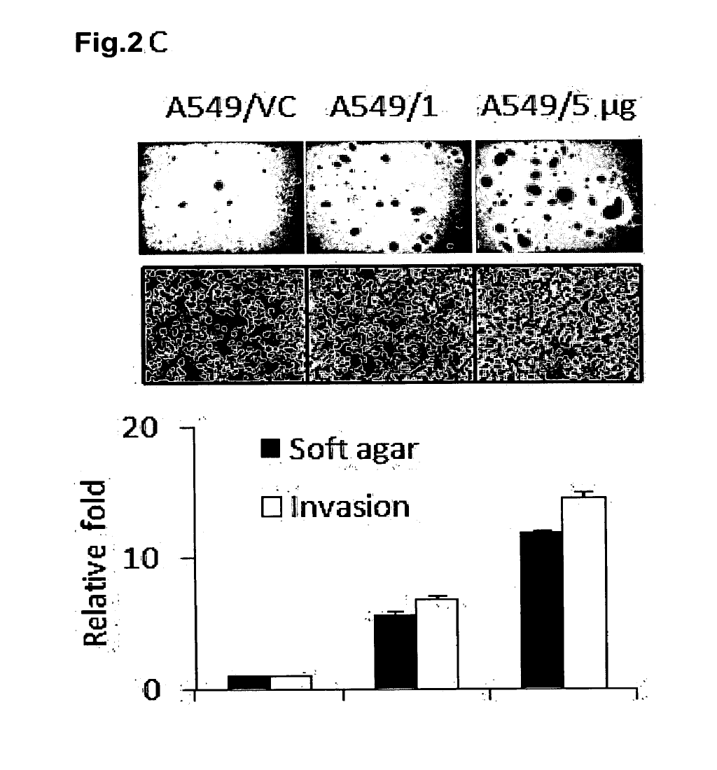

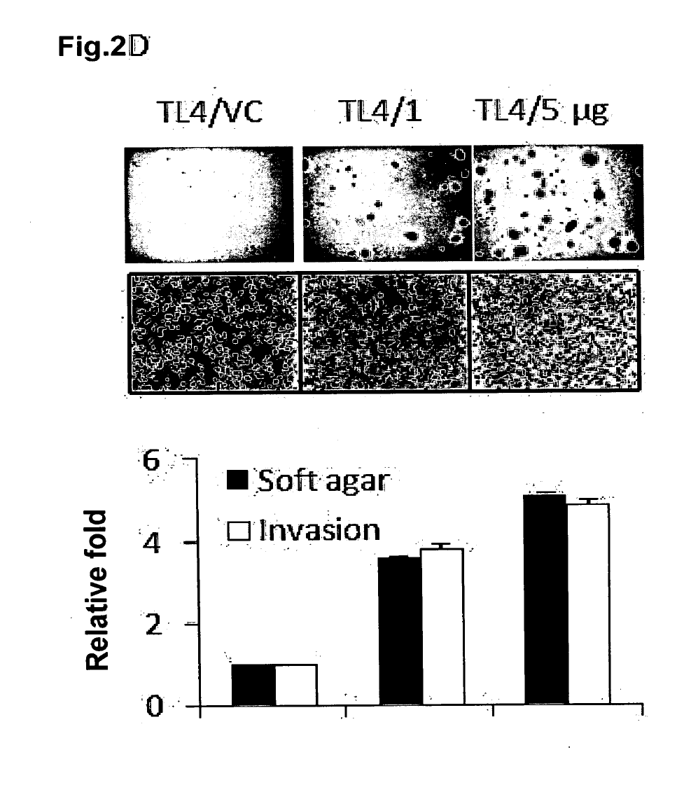

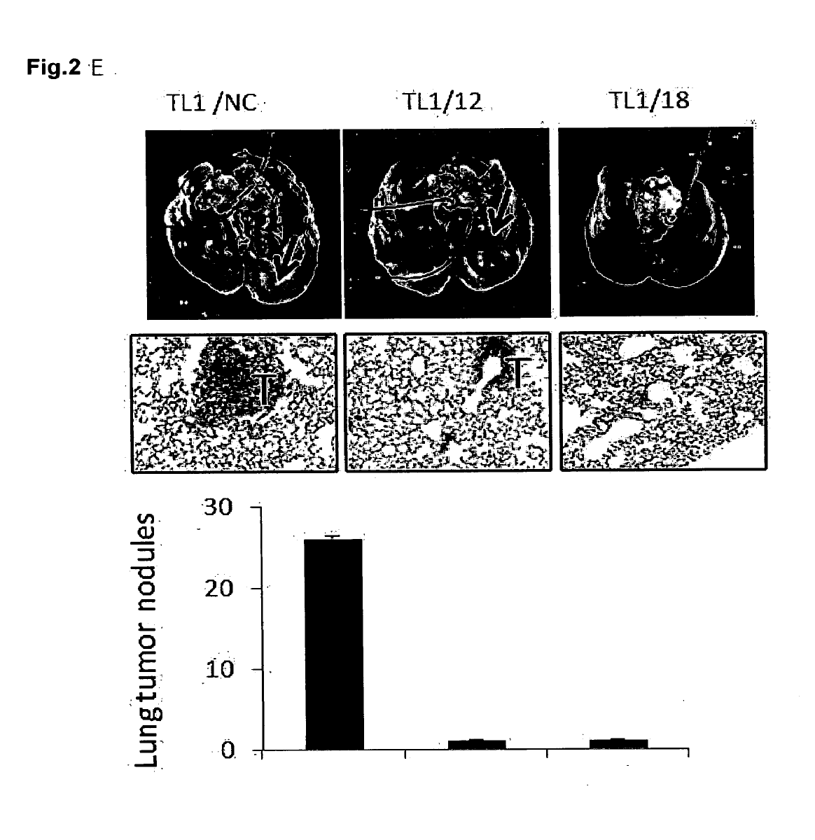

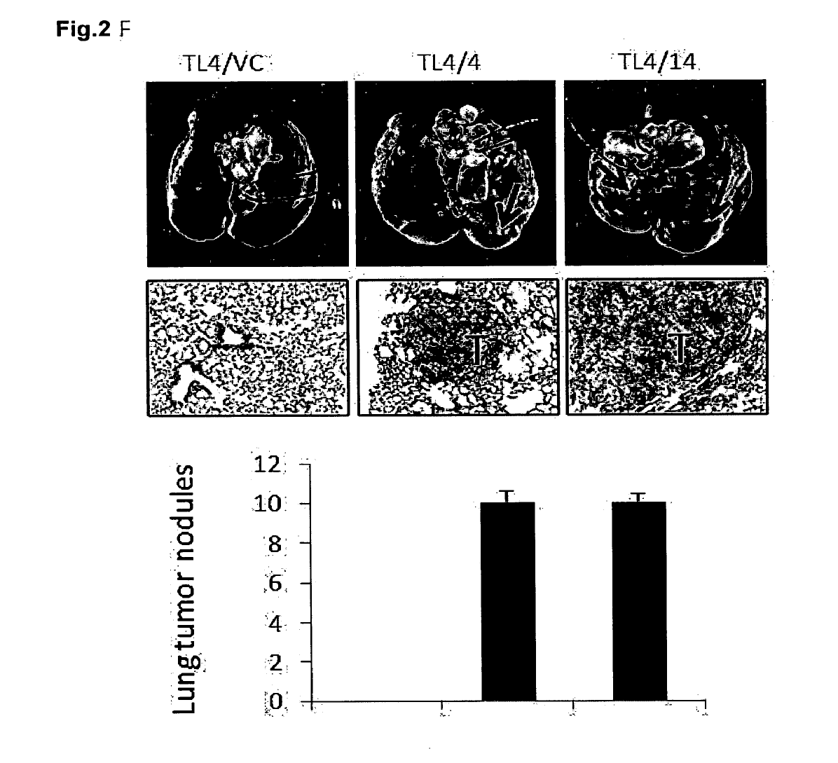

We examined whether PD-L1 could enhance cell growth and oncogenic potential, the doubling time, colony formation efficacy, Boyden chamber, and soft-agar assay were performed in PD-L1-knockdown TL-1 and TL-2 cells, and PD-L1-overexpression A549 and TL-4 cells compared to both cells with non-specific shRNA transfection (NC). The doubling time of PD-L1-knockdown TL-1 and TL-2 cells increased markedly compared to their NC cells (see FIG. 1A-1D). Conversely, the doubling time of PD-L1-overexpression A549 and TL-4 cells were shorter than both cells with empty vector transfection (VC) (see FIG. 1E-1H). Colony formation assay further confirmed the cell proliferation modulated by PD-L1-knockdown and -overexpression in these cells (see FIG. 1). Boyden Chamber and anchorage-independent soft-agar colony formation assay indicated that efficacy of the invasiveness and anchorage-independent soft-agar colony formation were decreased in PD-L1-knockdown cells and increased in PD-L1-overexpressng cells in a dose-dependent manner compared to their NC and VC cells (see FIG. 2A-2D). These results clearly indicate that PD-L1 promotes the cell proliferation and oncogenic potential in NSCLC cells via the autocrine loop.

### Example 2 PD-L1 Promotes Xenograft Metastatic Lung Tumor Formation in Nude Mice

We explored whether PD-L1 could promote xenograft metastatic tumor formation in nude mice, PD-L1-knockdown TL-1 stable clone #1 and #2 (TL/#1 and TL1/#2) and PD-L1-overexpression TL-4 stable clone #1 and #2 (TL4/#1 and TL4/#2) were established to inject into nude mice compared to those injected with TL1/NC and TL4/VC cells, respectively. Ten mice of each group were injected with the stable clones via tail vein. After 55 days, all mice were sacrificed and tumor burden in lung organ were measured and counted. Results showed that 10, 5 and 4 mice of the TL1/NC, TL1/#1 and TL/#2 group were found to have lung tumor nodules. The numbers of lung tumor nodules in the TL1/NC group were significantly higher than in the TL1/#1 and TL1/#2 group (see FIG. 2E). Conversely, 0, 7, and 10 mice in the TL4/VC, TL4/#1 and TL4/#2 groups were observed to possess lung tumor nodules; the numbers of lung tumor nodules in the TL4/#1 and TL/#2 group were significantly higher than in the TL4/VC group (see FIG. 2F). The tumor size of these lung tumor nodules in the group of TL1/NC, TL4/#1 and TL/#2 was significantly higher than in the group of TL1/#1, TL1/#2 and TL4/VC. These results clearly indicate that PD-L1 promotes xenograft metastatic lung tumor formation in nude mice (see FIG. 2).

Surprisingly, our data from cell and animal model indicated that PD-L1 expressed from lung cancer cells can directly promote the soft-agar growth, invasiveness, and metastatic xenograft tumor formation (see FIG. 1 and FIG. 2).

### Example 3 the Profiles of the Cell Cycle- and Metastatic-Related Genes in PD-L1-Knockdown TL-1 Cells

We examined which cell-cycle- and metastatic-related gene could be responsible for PD-L1-mediated cell proliferation and oncogenic potential, PD-L1-knockdown TL-1 stable clones#1 was used to evaluate the change of cell-cycle- and metastatic-related gene expression profiles by PCR array. As shown in Tables 1 and 2, more than 2 fold induction of 25 cell cycle- and 9 metastatic-related genes were observed in PD-L1-knockdown TL-1 cells compared to TL1/NC cells. Among these, three cell cycle-related genes (TGFβ1, p21, and p53) and two metastatic-related genes (SMAD4 and Maspin) were markedly elevated, but VEGF-C expression was significantly reduced in PD-L1-knockdown TL-1 cells. P21 and VEGF-C have been shown to be targeted by the TGFβ/SMAD4 pathway. Therefore, we suggest that p21 and VEGF-C might be involved in PD-L1-mediated tumor progression and metastasis via the TGFβ1/SMAD4 pathway.

TABLE 1
Cell cycle-related genes expressions in shPD-L1 cells were analyzed by
PCR array.

| | ShPDL1/NC | | ShPDL1/NC |
|---|---|---|---|
| Down-regulated genes | (Fold) | Up-regulated genes | (Fold) |
| ATM | 0.46 | TGFβ1 | 3.28 |
| ATR | 0.42 | p21 | 2.96 |
| cyclin A1 | 0.22 | p53 | 2.42 |
| cyclin A2 | 0.19 | | |
| cyclin E1 | 0.31 | | |
| cyclin E2 | 0.53 | | |
| cdk2 | 0.22 | | |
| cyclin D1 | 0.50 | | |
| cyclin D3 | 0.35 | | |
| cdk4 | 0.43 | | |
| cdk6 | 0.38 | | |
| cyclin B1 | 0.33 | | |
| cyclin B2 | 0.61 | | |
| cdc2 | 0.33 | | |
| cdc25A | 0.28 | | |
| E2F1 | 0.35 | | |
| E2F2 | 0.06 | | |
| HDAC2 | 0.65 | | |
| HDAC4 | 0.32 | | |
| RB | 0.58 | | |
| RAF1 | 0.52 | | |
| serin/threonine-protein | 0.54 | | |
| phosphatase 2A | | | |
| TWIST | 0.29 | | |
| PAI-1 | 0.45 | | |
| MAT1 | 0.45 | | |
| MAT2 | 0.21 | | |
| CXCR4 | 0.26 | | |

### Example 4 P21 and VEGF-C are Responsible for PD-L1-Mediated Soft-Agar Growth and Invasiveness Via the TGFβ1/SMAD4 Pathway

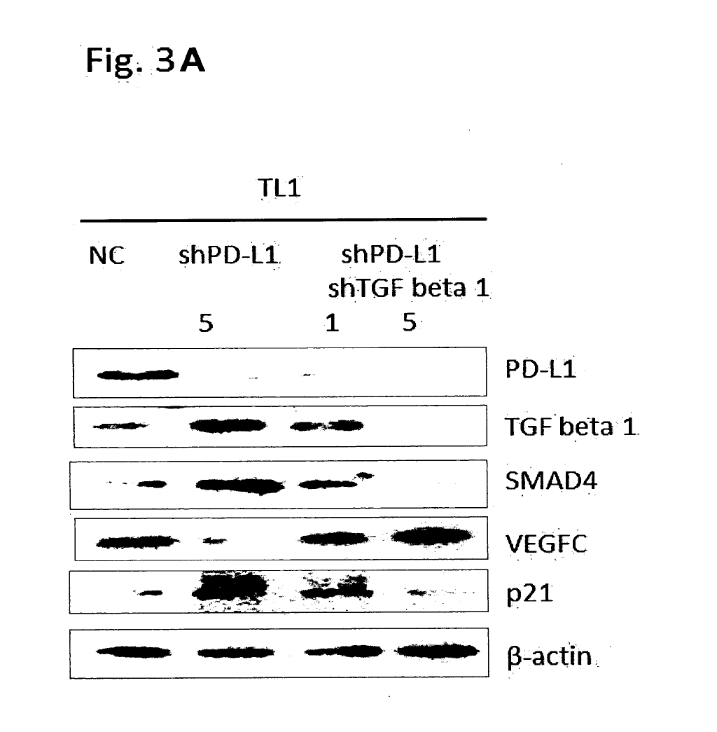

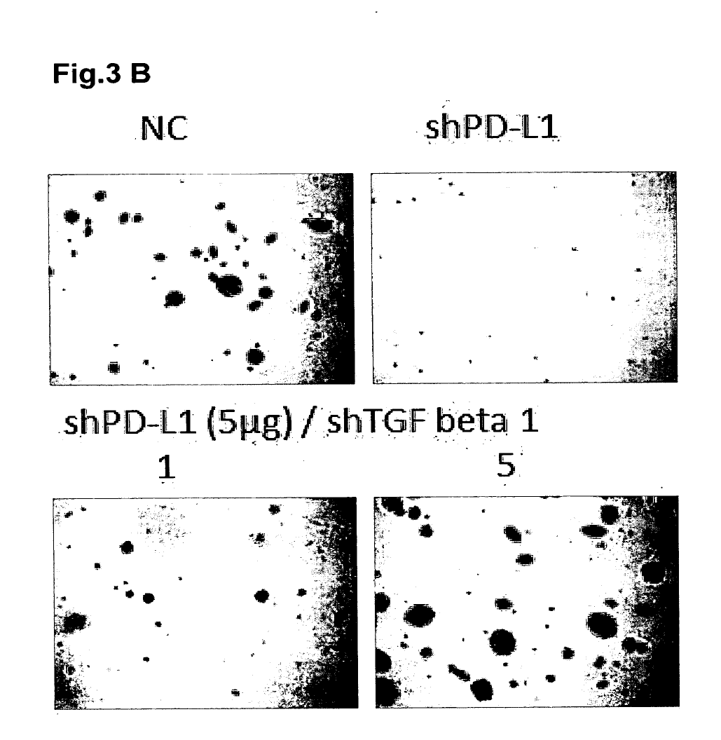

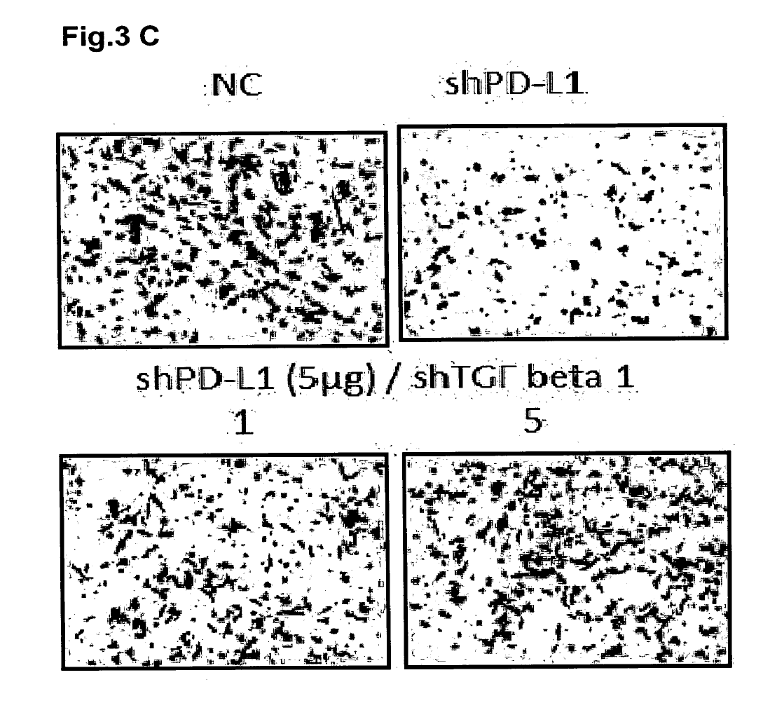

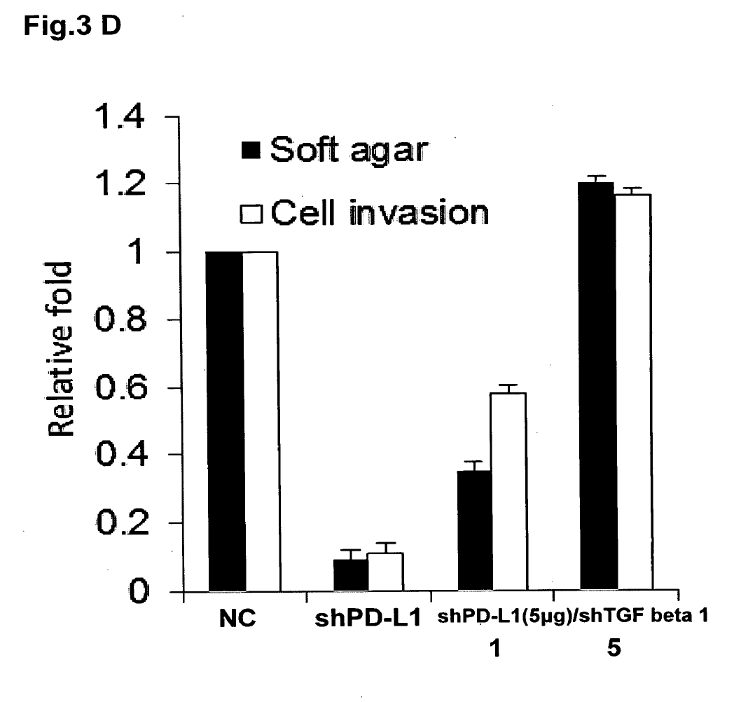

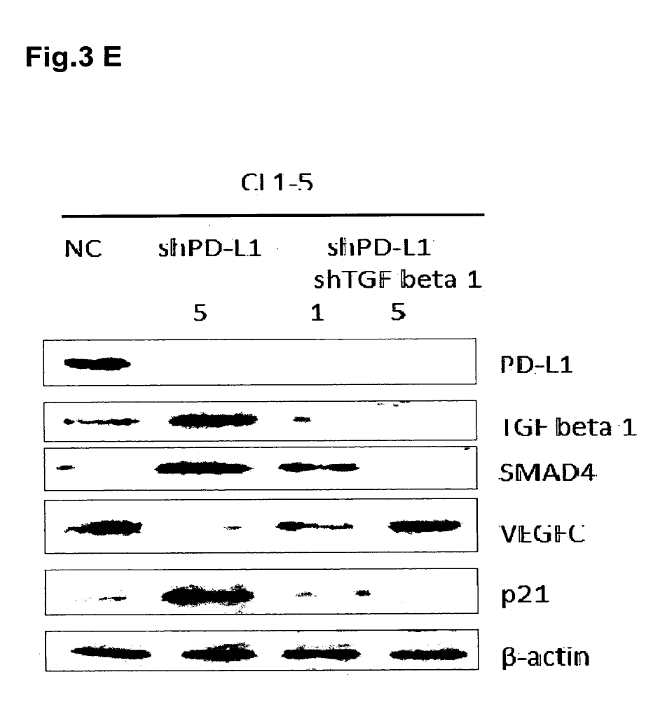

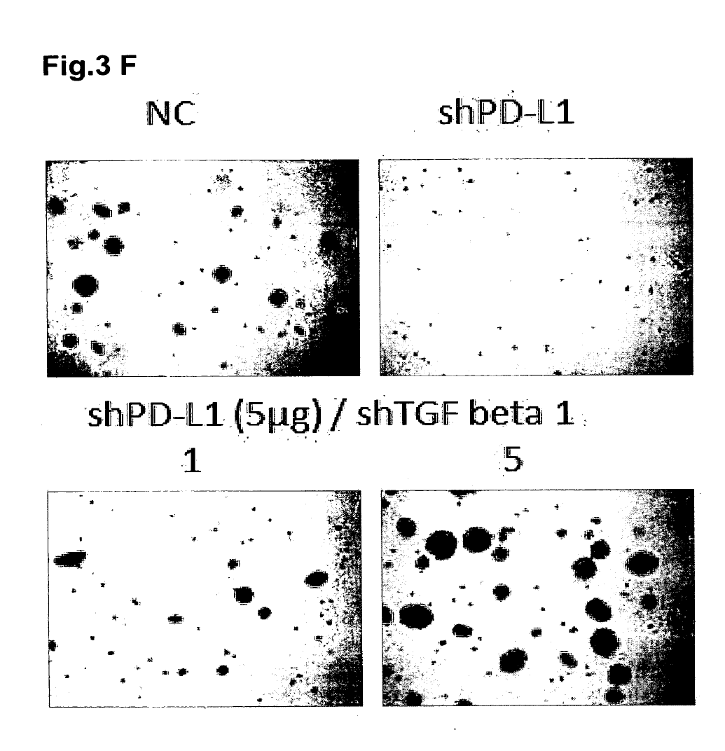

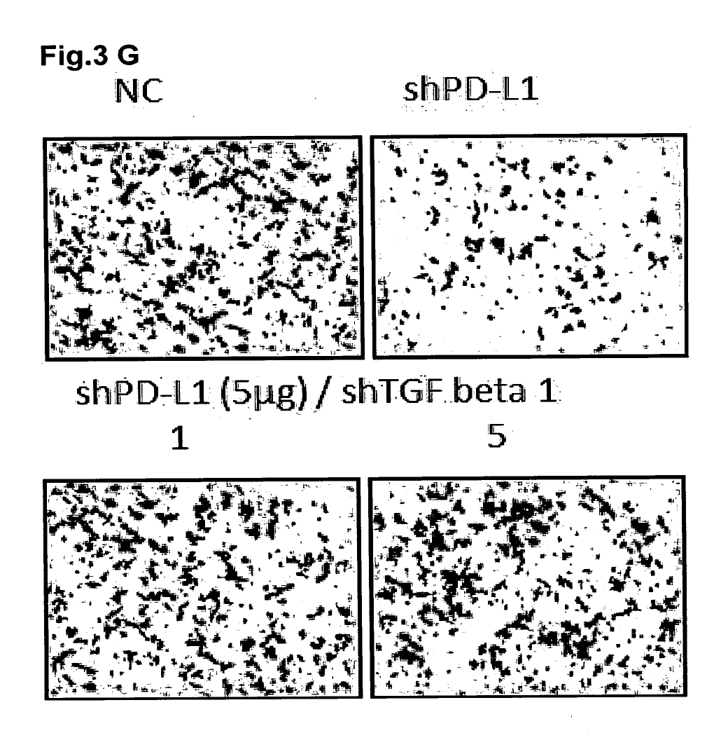

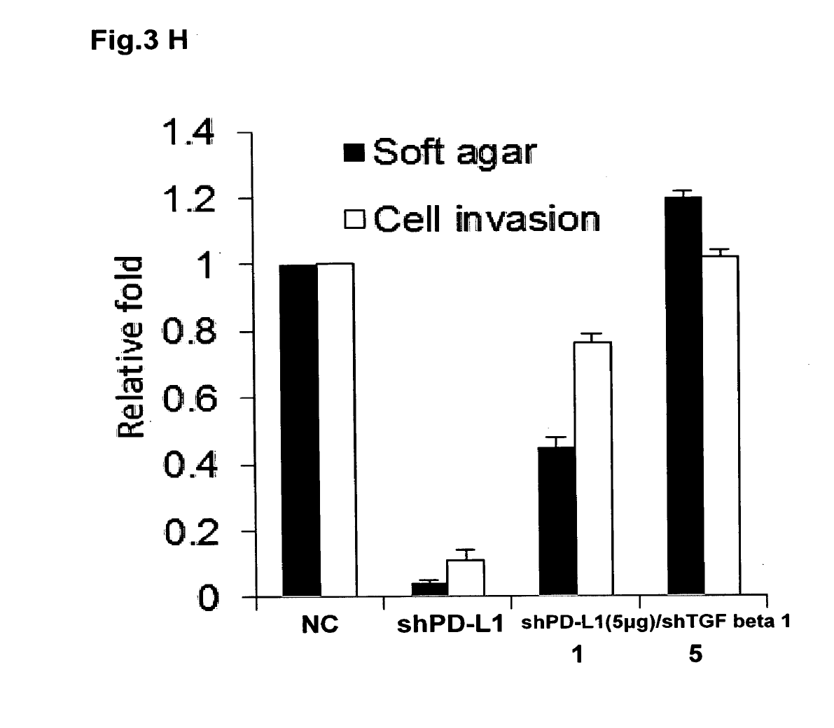

We explored the possibility that p21 and VEGF-C deregulated by PD-L1 could promote soft-agar growth and invasiveness via the TGFβ1/SMAD4 pathway. High PD-L1 expressing TL-1 and CL1-5 cells were collected to knockdown PD-L1 and then further silenced TGFβ1 in PD-L1-knockdown TL-1 and CL1-5 cells to examine whether p21, VEGF-C, TGFβ1, and SMAD4 expressions were changed by PD-L1-knockdown and further TGFβ1-silencing in both cells. Western blotting revealed that the expression of TGFβ1, SMAD4 and p21 were markedly elevated, but VEGF-C expression was significantly reduced by PD-L1-knockdown in TL-1 and CL1-5 cells compared to both NC cells. Interestingly, the expression of SMAD4, VEGF-C, and p21 were dose-dependently rescued by TGFβ1-silencing in PD-L1-knockdown TL-1 and CL1-5 cells. The representative colony growth on the soft agar plates and invasive cells on the Matrigel membranes in TL-1 and CL1-5 cells with PD-L1-knockdown or further TGFβ1-silencing are shown in FIG. 3. The efficacy of soft agar growth and invasiveness of both cells was dependent on the PD-L1-mediated the reduction of p21, TGFβ1, SMAD4 expression and elevation of VEGF-C expression in both cells. These results indicate that p21 and VEGF-C may be responsible for PD-L1-mediated soft-agar growth and invasiveness via the TGFβ1/SMAD4 pathway.

### Example 5 Relationships of PD-L1 mRNA Expression with Clinical Parameters in Lung Cancer Patients

PD-L1 mRNA expression levels in 223 lung tumors were evaluated by real-time PCR and the expression levels were ranged from 0.1231 to 8374.391. The median value (9.08237) was used as a cut-off point to categorize tumors into high and low PD-L1 mRNA level groups. We examined whether PD-L1 mRNA expressions in lung tumors could be associated with clinical-pathological parameters. As shown in Table 3, high PD-L1 mRNA level was more common in patients from female, nonsmokers, adenocarcinoma and late stage (III) compared with patients from male, smokers, squamous cell carcinoma and early stage (I, II) (70% vs. 41%, P<0.001 for genders; 59% vs. 39%, P=0.003 for smoking status; 66% vs. 34%, P<0.001 for histology type). These results suggest that PD-L1 overexpression may play more important role in lung tumor progression and metastasis of female, nonsmokers, and adenocarcinoma patients. Most importantly, PD-L1 mRNA expressions in HPV 16/18 E6 positive tumors were significantly higher than in HPV 16/18 E6 negative tumors (P<0.001). These results suggested that PD-L1 may play a role in HPV-infected lung tumorigenesis.

TABLE 3
Relationship between PDL-1, TGFbeta1, VEGF-C protein expression and clinical-
pathological parameters in lung cancer patients.

| | | PD-LI protein | | | TGFbetal protein | | | VEGF-C protein | | |
|---|---|---|---|---|---|---|---|---|---|---|
| Parameters | Case no. | Negative (%) | Positive (%) | p | Negative (%) | Positive (%) | p | Negative (%) | Positive (%) | p |
| | 140 | 62 (87.9) | 78 (42.1) | | 81 (87.9) | 59 (42.1) | | 77 (55.0) | 63 (45.0) | |
| Age | | | | | | | | | | |
| ≤65 | 66 | 28 (42.4) | 38 (57.6) | 0.675 | 36 (54.5) | 30 (45.5) | 0.454 | 35 (53.0) | 31 (47.0) | 0.658 |
| >65 | 74 | 34 (45.9) | 40 (54.1) | | 42 (56.8) | 29 (39.2) | | 42 (56.8) | 32 (43.2) | |
| Gender | | | | | | | | | | |
| Female | 45 | 18 (40.0) | 27 (60.0) | 0.482 | 28 (62.2) | 17 (37.8) | 0.733 | 22 (48.9) | 23 (51.1) | 0.317 |
| Male | 95 | 44 (46.3) | 51 (53.7) | | 53 (55.8) | 42 (44.2) | | 55 (57.9) | 40 (42.1) | |
| Tumor type | | | | | | | | | | |
| AD | 78 | 29 (37.2) | 49 (62.8) | 0.058 | 42 (53.8) | 36 (46.2) | 0.281 | 42 (53.8) | 36 (46.2) | 0.758 |
| SQ | 62 | 33 (53.2) | 29 (46.8) | | 39 (62.9) | 23 (37.1) | | 35 (56.5) | 27 (43.5) | |
| Smoking status | | | | | | | | | | |
| Nonsmoking | 83 | 36 (43.4) | 47(56.6) | 0.793 | 49 (59.0) | 34 (41.0) | 0.733 | 48 (53.2) | 35 (42.2) | 0.416 |
| Smoking | 57 | 26 (45.6) | 31 (54.4) | | 32 (56.1) | 25 (43.9) | | 32 (57.1) | 28 (49.1) | |
| Stage | | | | | | | | | | |
| I | 51 | 26 (51.0) | 25 (49.0) | 0.420 | 33 (64.7) | 18 (35.3) | 0.116 | 27 (52.9) | 24 (47.1) | 0.862 |
| II | 25 | 9 (36.0) | 16 (64.0) | | 10 (40.0) | 15 (60.0) | | 13 (52.0) | 12 (48.0) | |
| IV | 64 | 27 (42.2) | 37 (57.8) | | 38 (59.4) | 26 (40.6) | | 37 (57.8) | 27 (42.2) | |
| Tumor Size | | | | | | | | | | |
| T1 + T2 | 111 | 51 (45.9) | 60 (54.1) | 0.439 | 67 (60.4) | 44 (39.6) | 0.241 | 58 (52.3) | 53 (47.7) | 0.201 |
| T3 + T4 | 29 | 11 (37.9) | 18 (62.1) | | 14 (48.3) | 15 (51.7) | | 19 (65.5) | 10 (34.5) | |
| N | | | | | | | | | | |
| N0&N1 | 90 | 40 (44.4) | 50 (55.6) | 0.960 | 51 (56.7) | 39 (43.3) | 0.702 | 48 (53.3) | 42 (46.7) | 0.595 |
| N2&N3 | 50 | 22 (44.0) | 28 (56.0) | | 30 (60.0) | 20 (40.0) | | 29 (58.0) | 21 (42.0) | |
| PD-LI protein | | | | | | | | | | |
| Negative | 62 | | | | 28 (45.1) | 34 (54.9) | 0.007 | 49 (79.0) | 13 (21.0) | <0.001 |
| Positive | 78 | | | | 53 (67.9) | 25 (32.1) | | 28 (35.9) | 50 (64.1) | |
| TGFbetal | | | | | | | | | | |
| protein | | | | | | | | | | |
| Negative | 81 | | | | | | | 34 (42.0) | 47 (58.0) | <0.001 |
| Positive | 59 | | | | | | | 43 (72.9) | 16 (27.1) | |

### Example 6 High PD-L1 mRNA Levels May Independent Predict OS and RFS in Lung Cancer Patients

To verify whether PD-L1 mRNA level was associated with OS and RFS in NSCLC patients, Kaplain-Meier survival and multivariate Cox regression analysis were used to statistically analyze. Median follow-up period of 27.2 months, 86 patients relapsed including 19 had local recurrence, 46 had distance metastasis, and 21 had local and distance metastasis. None of the patients received adjuvant chemotherapy before surgical therapy. Kaplain-Meier analysis showed that patients with high PD-L1 mRNA level had shorter OS and RFS than those with low PD-L1 mRNA levels (Table 4). Multivariate Cox regression analysis showed that the hazard ratio (HR) for OS and RFS in patients with high PD-L1 mRNA levels were 2.54 (OS) and 2.06 (RFS) times those with low PD-L1 mRNA levels, respectively (95% CI, 1.76 to 3.66, P<0.001 for OS; HR, 2.06, 95% CI, 1.44 to 2.93, P<0.001 for RFS, Table 4). More interestingly, E6 positive patients with high PD-L1 expression had the poorest OS and RFS among the 4 groups (see Table 4). These results suggest that induction of PD-L1 may regulate by E6 to promote malignancy in patients and leads to poor OS and RFS.

TABLE 4
Cox regression analysis for the influence of HPV16/18 E6, PD-L1 and combined effects
on OS and RFS in lung cancer patients.

| | | OS | | | | | RFS | | | | |
|---|---|---|---|---|---|---|---|---|---|---|---|
| | | | Survival | | | | | Survival | | | |
| | | Median | rate at 5 | | | | Median | rate at 5 | | | |
| | N | months | year (%) | HR* | 95% CI | P | months | year (%) | HR* | 95% CI | P |
| E6 protein | | | | | | | | | | | |
| Negative | 163 | 30.6 | 39.9 | 1 | | | 23.9 | 28.0 | 1 | | |
| Positive | 60 | 26.4 | 18.3 | 1.32 | 0.92~1.91 | 0.130 | 16.8 | 15.0 | 1.39 | 0.97~1.97 | 0.066 |
| PD-L1 | | | | | | | | | | | |
| Low | 111 | 60.8 | 53.2 | 1 | | | 37.7 | 36.9 | 1 | | |
| High | 112 | 18.9 | 15.2 | 2.69 | 1.83~3.93 | <0.001 | 13.8 | 13.4 | 2.01 | 1.40~2.85 | <0.001 |
| PD-L1/HPV E6 | | | | | | | | | | | |
| −/− | 92 | 60.8 | 52.2 | 1 | | | 40.0 | 38.0 | 1 | | |
| −/+ | 13 | — | 53.8 | 1.06 | 0.52~2.15 | 0.860 | 78.7 | 46.2 | 0.77 | 0.34~1.70 | 0.521 |
| +/− | 71 | 19.5 | 23.9 | 1.41 | 1.15~1.74 | 0.001 | 14.1 | 16.9 | 1.32 | 1.08~1.61 | 0.026 |
| +/+ | 47 | 25.9 | 8.5 | 2.19 | 1.48~3.26 | <0.001 | 14.8 | 6.4 | 1.84 | 1.29~2.38 | 0.001 |

All HR were adjusted for age, gender, smoking status, stage and type.

What is claimed is:

1. A method for treating and/or preventing a tumor growth, invasion and/or metastasis in a subject, comprising administering a PD-1 expression inhibitor to a subject to knockdown an expression of PD-L1 in the subject via an autocrine loop.

2. The method of claim 1, further comprising a step of administrating a TGF-β1 expression inhibitor.

3. The method of claim 1, further comprising a step of administrating a VEGF expression inhibitor.

4. The method of claim 1, wherein tumor invasion or metastasis can be inhibited.

5. The method of claim 1, wherein the tumor is a virus-associated tumor.

6. The method of claim 5, wherein the virus is HPV, HIV, EBV HBV, CMV or HCV.

7. The method of claim 1, wherein the PD-L1 expression inhibitor is a small interfering RNA (siRNA) of PD-L1, a small hairpin (sh)RNA of PD-L1 or an antisense RNA of PD-L1.

8. The method of claim 1, wherein the subject is a relapsed or refractory subject.

9. The method of claim 1, wherein the subject is a mammal.

10. The method of claim 1, wherein the subject is a human.

11. The method of claim 1, wherein the tumor is HPV-, HIV-, HCV-, EBV-, CMV or HBV-associated cancer, renal cancer, colon cancer, anal cancer, reproductive organ cancer, uterine cancer, ovarian cancer, endometrial cancer, cervical cancer, vaginal cancer, vulvar cancer, breast cancer, kidney cancer, pancreatic cancer, colon cancer, large bowel cancer, lung cancer, liver cancer, brain tumor, gastric cancer, uterine cervix cancer, ovary cancer, prostate cancer, urinary bladder cancer, esophageal cancer, leukemia, lymphoma, fibrosarcoma, mastocytoma, or melanoma.

12. The method of claim 11, wherein the tumor is leukemia, anal cancer, vulvar cancer, vaginal cancer, penile cancer, cervical cancer, head and neck cancer such as oropharyngeal cancer and cancer of the oral cavity, lung cancer, colon cancer, non-melanoma skin cancer, HPV-associated cancers, or liver cancer.

13. The method of claim 1, wherein the tumor is a non-small-cell lung carcinoma (NSCLC) or a HPV-associated cancer.

14. A pharmaceutical combination, comprising a PD-L1 expression inhibitor in combination with a TGF-β1 expression inhibitor or VEGF expression inhibitor.

15. The pharmaceutical combination of claim 14, which further comprises a second anticancer agent or therapy.

16. The pharmaceutical combination of claim 14, wherein the PD-L1 expression inhibitor is a small interfering RNA (siRNA) of PD-L1, a small hairpin (sh)RNA of PD-L1, an antisense RNA of PD-L1, an anti-PD-L1 antibody or an antigen-binding fragment thereof.

17. The pharmaceutical combination of claim 16, wherein the anti-PD-L1 antibody is chimeric, humanized, composite, human antibody or bispecific antibody.


SEQUENCE LISTING (5 sequences)

SEQ ID NO: 1 - DNA, 20 bp, artificial
  synthetic oligonucleotide
acctgacctgccgtctagaa

SEQ ID NO: 2 - DNA, 20 bp, artificial
  synthetic oligonucleotide
tccaccaccctgttgctgta

SEQ ID NO: 3 - DNA, 22 bp, artificial
  synthetic oligonucleotide
gtgagcgatggaacttcgactt

SEQ ID NO: 4 - DNA, 22 bp, artificial
  synthetic oligonucleotide
ggcgtttggagtggtagaaatc

SEQ ID NO: 5 - DNA, 21 bp, artificial
  synthetic oligonucleotide
gctgcactaattgtctattgg